(19) United States
(12) Patent Application Publication
(10) Pub. No.: US 2020/0323358 A1
(43) Pub. Date: Oct. 15, 2020

(54) Absorbent Mat with Wings Connected by Tear-Open Sections

(71) Applicant: Teknoweb Materials S.R.L., Palazzo Pignano (IT)

(72) Inventors: Fabio Zampollo, Crema (IT); Ivano Gagliardi, Pescara (IT)

(21) Appl. No.: 16/096,309

(22) PCT Filed: Apr. 28, 2017
(86) PCT No.: PCT/EP2017/060189

§ 371 Date: Oct. 25, 2018

(30) Foreign Application Priority Data
Apr. 28, 2016 (GB) 1607362.9
Aug. 3, 2016 (GB) 1613422.3

(51) Int. Cl. A47C 31/10; A61F 5/48; B32B 5/02; B32B 5/26

(52) U.S. Cl. CPC A47C 31/105; A61F 5/485; A47G 9/0238; B32B 5/26; B32B 5/022

(57) ABSTRACT

The present invention is an absorbent mat, such as a bed pad or a change mat, e.g. for protecting an underlying support from soiling, as well as method for the formation of such a pad or mat. The mat has laterally extending wings, that are connected to each other by a tear-open section in a manufacturing configuration, that is opened to create the use configuration.

### FIELD OF THE INVENTION

The present invention is an absorbent mat, such as a bed pad or a change mat, e.g. for protecting an underlying support from soiling, as well as method for the formation of such a pad or mat.

### BACKGROUND

Mats or pads to prevent soiling of a support on which such mats are placed, are well known in the art, see e.g. U.S. Pat. No. 4,097,943 describing an under-pad for a bed comprising an absorbent layer, which is enveloped between a liquid permeable and an impermeable layer, or WO20060431151 describing a baby changing mat. It is also known to apply laterally extending straps, or wings or panels to allow for better fixation of the pad or mat, see U.S. Pat. No. 5,221,273A, describing a pad with laterally extending straps, or U.S. Pat. No. 4,391,010, describing lateral wings, that may be wrapped around and underneath the mattress supporting the pad, or EP2967669A1, describing a pad with one or more lateral wing(s) that can be releasably connected to the absorbent structure of the pad, such that this structure may be discarded upon soiling, whilst the lateral wings might be continued to be used or even washed for a clean re-use.

However, in particular larger sized wings are difficult to handle both in the manufacturing process as well as upon use of the pads, such as by a caregiver.

Further it may be desirable that the user oriented surface of the absorbent structure is maintained clean and protected from any soiling prior to the application.

Even further, it might be desirable to achieve good liquid control, in particular good side leakage control, with a minimum of materials used.

These challenges—either individually or in combination—are addressed by the present invention.

### SUMMARY

In a first aspect, the present invention is an absorbent mat for being positioned on a support as may be a mattress, for a person as a user, wherein the absorbent mat exhibits in Cartesian coordinates a longitudinal or length (x-) direction, a width (y-) direction, a thickness (z-) direction perpendicular thereto, and a longitudinally extending center line.

The absorbent mat comprises an absorbent composite comprising
(i) an absorbent structure exhibiting a length, width, and thickness direction aligned with the length, width and thickness direction of the absorbent mat, and having a thickness extension substantially smaller than its length and width extension, further a first surface adapted to be oriented towards a user during use, and a second, opposite surface;
(ii) a liquid permeable topsheet positioned on the first surface of the absorbent structure;
(iii) a liquid impermeable backsheet positioned on the second surface of the absorbent structure z-directionally opposite of the topsheet;
   whereby preferably the topsheet and the backsheet are enveloping the absorbent structure in MD, CD, and thickness direction.

The absorbent composite exhibits a first and a second longitudinally extending lateral side margin region positioned oppositely of the longitudinally extending center line of the absorbent mat.

The absorbent mat further comprises a 1^{st} and a 2^{nd} wing.

The 1^{st} wing exhibits a first 1^{st} wing side margin region in an at least partly overlapping positioning with the first longitudinally extending lateral side margin region of the absorbent composite, and a longitudinally extending second 1^{st} wing side margin outside of the first 1^{st} wing side margin region. The 1^{st} wing is connected by a connection in the first 1^{st} wing side margin region to the longitudinally extending first lateral side margin region of the absorbent composite.

The 2^{nd} wing exhibits a first 2^{nd} wing side margin region in an at least partly overlapping positioning with the second longitudinally extending lateral side margin region of the absorbent composite, and a longitudinally extending second 2^{nd} wing side margin outside of the first 2^{nd} wing side margin region. The 2^{nd} wing is further connected by a connection in the first 2^{nd} wing side margin region to the longitudinally extending second lateral side margin region of the absorbent composite.

The absorbent mat is adapted to be transformed from a manufacturing configuration into a use configuration.

In the manufacturing configuration at least a portion of the 1^{st} and the 2^{nd} wing extends y-directionally from the first and second side margin regions towards the longitudinally extending centerline of the absorbent mat, respectively, and the second 1^{st} wing side margin and the second 2^{nd} wing side margin are connected to each other along a tear-open section extending essentially parallel to the longitudinal centerline.

In the use configuration, the 1^{st} and the 2^{nd} wings are separated from each other along the tear-open section and at least portions of the second 1^{st} wing side margin and the second 2^{nd} wing side margin are positioned (a) on the backsheet side of the absorbent composite, or (b) laterally outwardly of the first and second longitudinally extending lateral side margin regions, respectively.

The tear-open section may be a zone of weakness, extending generally in the length direction, and is more preferably a straight or curved perforation line. The connecting of the wings to the absorbent composite may be selected from the group consisting of adhesive, heat-, or ultrasonic bonding, and may be executed as line(s), dots, or regions. In a particular execution, the absorbent composite is pre-formed.

In the manufacturing configuration the tear-open section of the wings is positioned on the topsheet side or on the backsheet side of the absorbent composite.

The wings may be zig-zag or leporello folded, and optionally comprise a fold fixation for such folds, preferably an adhesive dot or line.

The wings may be connected to the absorbent composite in the longitudinally extending first 1^{st} wing side margin region and the first 2^{nd} wing side margin region to the topsheet of the absorbent composite. The wings may be adapted to form a side seal wrapping of the absorbent composite in the use configuration. Preferably, the wings are essentially made from liquid impermeable but air permeable materials, preferably hydrophobic nonwoven. In a second aspect, the present invention is a process for the manufacture of an absorbent mat, which comprises an absorbent structure enveloped between a topsheet and a backsheet and wings, and which exhibits a length, width, and thickness direction, and a longitudinally extending centerline.

The process exhibits a machine direction essentially aligned with the length direction of the absorbent mat and comprises the following steps:
a) providing
   as materials
      an absorbent structure, preferably an absorbent web material,
      topsheet and backsheet web material,
      a web of wing material for forming a 1^{st} and a 2^{nd} wing,
   and as equipment
      web supply and guide means,
      means for machine-directionally partly separating webs, preferably a perforation
      cutting device,
      means for cross-directionally separating webs,
      connecting means;
b) enveloping the absorbent structure, preferably separated pieces of the absorbent web material, between the topsheet and the backsheet, thereby forming an essentially continuous web of absorbent composites exhibiting a first and a second longitudinally extending lateral side margin region positioned opposite of the longitudinally extending centerline;
c) creating a tear-open section generally parallel to the machine direction to the web of wing material, the tear-open section delimiting the 1^{st} and the 2^{nd} wing;
d) selecting at least one of
   the web of wing material and
   a web comprising the absorbent composite material
   as a continuous base web;
e) optionally separating essentially along a cross machine direction one of
   (i) the web of wing material and
   (ii) a web comprising the absorbent composite material into machine-directionally individualized pieces;
f) feeding the selected continuous base web(s) and the individualized pieces, if present, to a mat combination unit;
g) positioning the selected continuous base web(s) and the individual pieces, if present, in the mat combination unit
   such that the tear-open section of the web of wing material overlays at least portions of the topsheet or the backsheet, preferably of the topsheet, and
   such that the 1^{st} and the 2^{nd} wings extend from the tear-open section laterally outwardly into the longitudinally extending side margin regions, respectively, of the absorbent composite;
h) connecting the wings to the absorbent composite in the longitudinally extending side margin regions of the absorbent composite,
   wherein the connecting is preferably executed by the applying adhesives or one or more energy sources selected from the group consisting of heat, pressure, friction, and ultrasonics;
i) separating the continuous base web into the absorbent mats, optionally executing further processing steps, preferably comprising folding, and optionally comprising packaging,
   wherein the tear-open section of the wings remains essentially intact.

Optionally, in the steps g) and h) the wings are positioned on and connected to the topsheet of the absorbent composite.

In yet a further aspect, the present invention is a method of applying an absorbent mat to a support, whereby method comprising the steps of separating the 1^{st} and the 2^{nd} wing from each other along a tear-open section and optionally at least partly wrapping the support by the wings.

### BRIEF DESCRIPTION OF THE FIGURES

FIG. 1 depicts schematically and generally an absorbent mat as it may be arranged on a support.

FIG. 2 depicts schematically and generally an absorbent composite as may be used in an absorbent mat according to the present invention.

FIG. 3A depicts schematically a cross-sectional view of features of a mat according to the present invention in a use configuration.

FIG. 3B depicts schematically a perspective view of an absorbent mat according to the present invention both in a manufacturing and a use configuration.

FIG. 3C depicts schematically a perspective view of a further execution of an absorbent mat according to the present invention both in a manufacturing and a use configuration.

FIG. 3D depicts schematically a further arrangement of an absorbent mat in a use configuration on a flat support.

FIG. 4 A and B depict schematically two executions of an absorbent composite according to the present invention in their manufacturing configuration.

FIGS. 5 to 10 depict schematically various executions of the present invention, each in their manufacturing configuration (Fig. “n.A”) and their use configuration (Fig. “n.B”).

FIG. 11 A to C depict schematically further executions of the present invention with folded wings.

FIG. 12 A and B depict schematically a further execution of the present invention with a releasable in use wing-to-wing connection.

FIG. 13 is a schematic presentation for a process according to the present invention.

In these figures, dimensions are not necessarily to scale. Same numerals in different figures refer to identical or equivalent features.

### DETAILED DESCRIPTION

In a first aspect, the present invention is a disposable absorbent mat or pad comprising an absorbent composite and wings, in a second aspect, the present invention relates to the making of such an article, and in even a further aspect to the use of such an article.

The term “absorbent mat” is often also used interchangeably with the term “absorbent pad” and refers in the present context to a generally flat structure, though not necessarily with an even thickness, that is adapted to absorb liquid and that may prevent a support on which such a mat is positioned from getting soiled or wetted. An absorbent mat may be used as a bed pad for preventing soiling on a mattress or other bedding items like bed sheets or fitted sheets from bodily exudates of a person positioned there on, such as when the person is not wearing other protective clothing like a diaper, or when such clothing is overloaded or poorly fitting so as to leak at receipt of the bodily exudates of the wearer, as can be without any limitation any of faeces, urine, menses, wound secrets, but also saliva, or sweat. However, also an inverse scenario is considered to be within the present context, such as when a baby change mat is put on a ground to protect the baby from being soiled during diaper changes

Within the present context, reference is made to a Cartesian coordinate system wherein the absorbent mat is exhibiting a length, longitudinal or x-direction, a width, cross-directional or y-direction and a thickness or z-direction perpendicular thereto.

When the absorbent mat is put on a support which is typically described by length and width, such as a bed or a mattress of a bed, the x-, y-, and consequently also z-direction of the absorbent mat will be understood to be aligned with this direction. Typically, the manufacturing direction for such an article will be that the machine direction of the process or equipment is aligned with length direction of the article, in particular of the absorbent composite. During use, the wings extend laterally outwardly, i.e. away from the longitudinally extending centerline of the article, and/or downwardly, i.e. away from the user. Of course, such an article may also comprise flaps that extend from the longitudinal ends, but unless expressly referred to, wings in the present context are considered the ones positioned laterally or sideways of the absorbent composite.

When the support onto which an absorbent mat according to the present invention is placed has less clearly defined length and width orientation, such as when considering a baby change mat that may be placed on a table or even on the ground, the term length or longitudinal refers to the direction that the article had during its manufacture and that a skilled person will be readily able to determine such as from material properties or other characteristics, such as glue beads.

The term “disposable” as used herein means when referring to an absorbent mat that it is intended to be discarded in an environmentally considerate manner after use, which may be a single use or may be several uses such as when during the first use no or no substantial soiling is occurring. Thus, the present invention is not directed towards mats or parts thereof that are re-usable, e.g. by washing or laundering.

“Comprise,” “comprising,” and “comprises” are open ended terms, each specifying the presence of the feature that follows, but does not preclude the presence of other features. The words “typically”, “normally”, “advantageously” and the likes also qualify features which are not intended to limit the scope of the claims unless specifically indicated to do so.

An absorbent composite comprises an absorbent structure as well as a liquid permeable cover- or topsheet intended to be oriented towards a user during use, and a liquid impermeable backsheet opposite to the topsheet. Typically, the absorbent structure, the topsheet and the backsheet will be different and separate materials, however it is also considered that for certain embodiment these may be delivered as a unitary premanufactured material with certain portions thereof providing the respective functionality.

An absorbent structure can comprise various absorbent materials as well known in the art, such as woven material, non-woven material, airlaid material that may comprise absorbent fibers such as cellulosic fibers or absorbent man-made fibers, or binder fibers, but also particulate material such as without limitation so called well known superabsorbent polymer (SAP) materials. The term absorbent refers to the capability of receive and preferably retain liquids, often aqueous body exudates, within voids of the structure, such as in inter-fiber interstices, or within the materials, such as in cellulosic fibers upon swelling, or in the SAP-network.

The absorbent composite is typically the component of an absorbent mat which comprises all, or at least the majority of the liquid absorbent capacity, preferably fibrous in nature and/or with a superabsorbent material and which has the highest absorbent capacity of all the components of the absorbent mat. The absorbent composite preferably has sufficient internal structural integrity and strength so as to withstand the foreseeable in-use conditions. Thus, the absorbent composite may comprise strength providing outer topsheet and backsheet materials, and/or the absorbent structure between these exhibits sufficient internal strength such as by comprising binder, such as binder fibers that can be activated by heat.

Preferably, the structural integrity of the absorbent composite is not negatively affected by moisture.

The absorbent composite may have an overall length of from about 500 mm to about 1000 and in the manufacturing configuration an overall width of from about 500 mm to about 1000 mm. It may exhibit a thickness of from about 0.5 mm to about 10 mm or more. The absorbent structure of the absorbent composite may exhibit a basis weight of from about 40 g/m² to about 200 g/m²; as may be determined according to EDANA NWSP 130.1R0. Within the present context, an absorbent mat further comprises wings, that are connected to the longitudinally extending lateral margins of the absorbent composite.

The term “wing” as used herein is a panel made of a material selected from the group consisting of a woven fabric, a nonwoven web, a film, a tissue and combinations thereof and that preferably shows a good compatibility with the skin.

A wing is preferably made from essentially non-elastic material and non-extensible material, as can be determined according to the instructions in the method section herein below, from a handling but also from a cost point of view. However, for certain executions a wing may exhibit a certain extensibility, so as to allow better adaptation to the support or even a certain elasticity. It is also contemplated, that a wing is composed x-y-directionally of different materials, such that a non-extensible stripe is combined with an extensible one. Preferably, a wing material exhibits a certain air-permeability or “breathability”, as may be determined according to the instructions herein below. Such materials may be so called breathable or microporous films, apertured films or the like. More preferably wings are made from nonwoven materials.

The term “nonwoven web” as used herein refers to a manufactured material, web, sheet or batt of directionally or randomly oriented fibers, bonded by friction, and/or cohesion and/or adhesion, excluding paper and products which are woven, knitted, tufted, stitch-bonded, incorporating binding yarns or filaments, or felted by wet milling, whether or not additionally needled. The fibers may be of natural or man-made origin.

Without intending any limitation, preferred non-woven materials may be made by processes as well known in the art, and from a strength point of view are preferably of the spunbonded type, optionally combined with meltblown layers. The total basis weights of a wing material may be more than about 5 g/m², though often the basis weight will be more than 10 g/m² and for certain applications requiring good strength properties even more than 20 g/m²; as may be determined according to EDANA NWSP 130.1R0. For softness and cost reasons, the wing materials exhibit typically a basis weight of less than 100 g/m², preferably less than 50 g/m² or even less than 30 g/m².

For the present application, it is preferred that the wing exhibits barrier properties for aqueous liquids.

Thus in case of the preferred execution of non-wovens, these are preferably hydrophobic nonwovens, such as when using polyolefin, such as polyethylene or even more preferred polypropylene as base resin. Optionally, wing materials may be made from two or multi-compound fibers.

A wing may have dimensions from 100 mm to 2000 mm along the longitudinal central axis of the absorbent mat or the absorbent composite and a width of from 100 mm to 1200 mm along the.

A wing may have a length extension essentially co-extensive with the absorbent composite, or may be shorter, or for certain executions even longer. The shape of a wing may be rectangular, though any other shape, such as trapezoidal or rounded may be used. Preferably, the wings are made without creating trim or waste material.

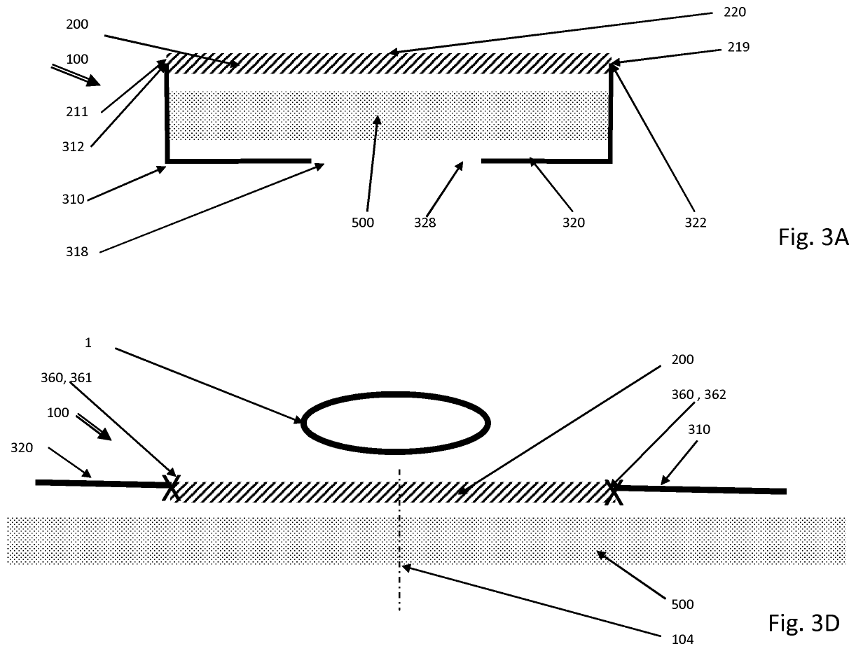

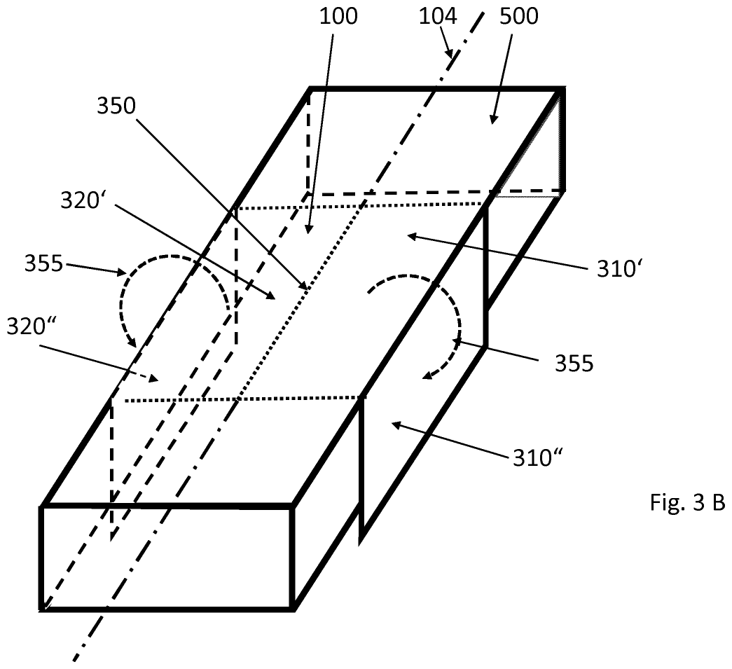

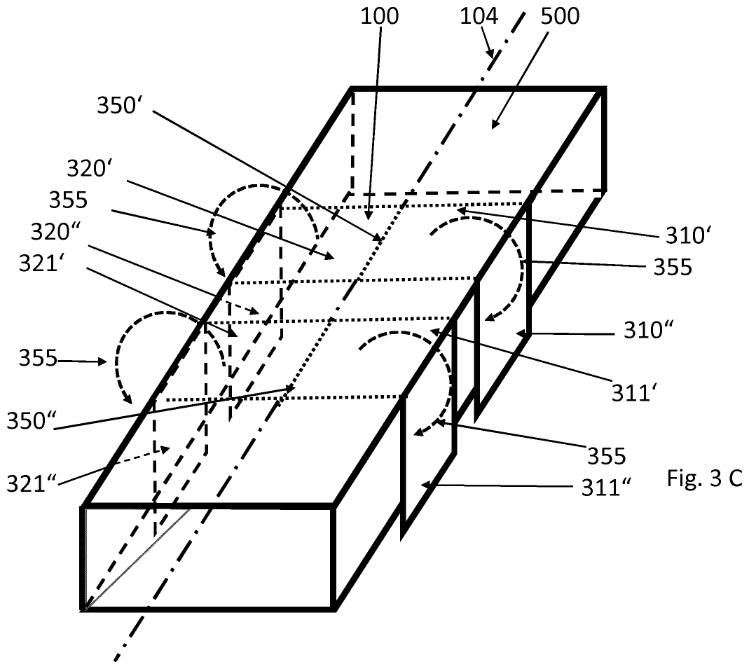

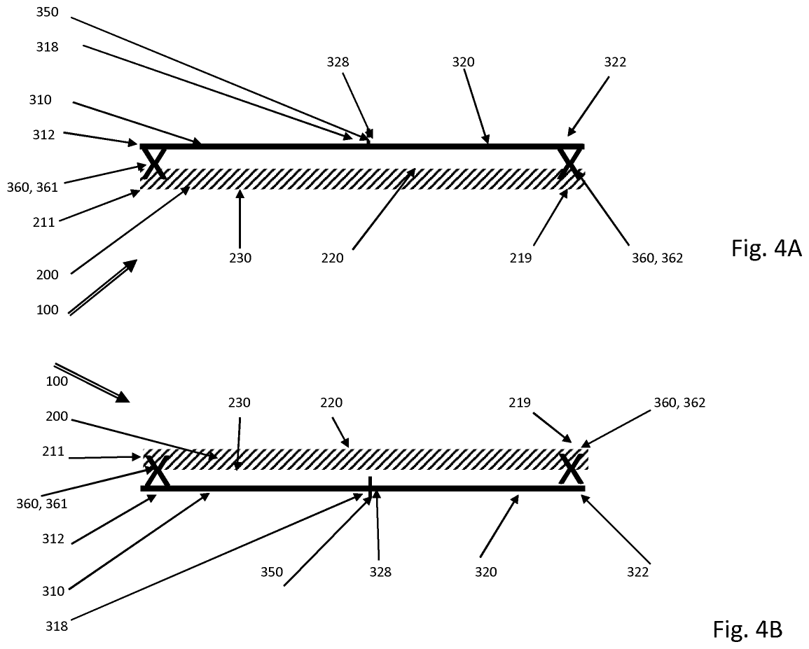

As indicated in FIG. 3 A, the absorbent mat (100) is formed by connecting the wings (300, resp. 310 and 320) to the absorbent composite (200). The connecting may be achieved by connections and ways to connect as are well known in the art, including without limitation, adhesives, heatbonding, and ultrasonic bonding means. The connecting can be a straight or curvilinear connecting line, such as a line of adhesives or heatbonding line, or a straight or curvilinear line of connecting points such as glue or heat bonding dots, or a connecting region, such as when applying a glue spray pattern with straight or curvilinear circumscribing lines. The wing material may exhibit a Bond strength between the wing and the absorbent composite of not less than about 1 N, when measured by Tensile Strength method EDANA WSP 401.0:RO. The connecting may also be achieved by a recloseable means, such as mechanical fastener, such as described in EP2967669A1 (P&G) to which express reference is made as far as this connecting is concerned.

Whilst for ease of manufacturing the side margins of the absorbent composite or the absorbent mat are preferably straight and parallel to the longitudinal centerline, it is also within the scope of the present invention that may have curved, for example undulating, shape or taper somewhat to create a trapezoidal shape.

It is an important aspect of the present invention that the wing and the absorbent composite are arranged in a particular manner that allows for a first configuration, as being prepared during manufacturing, to be converted by a user into a second, use configuration. This is explained in the following by referring to the respective figures, which should, however not be seen to limit the present invention to the specific executions shown.

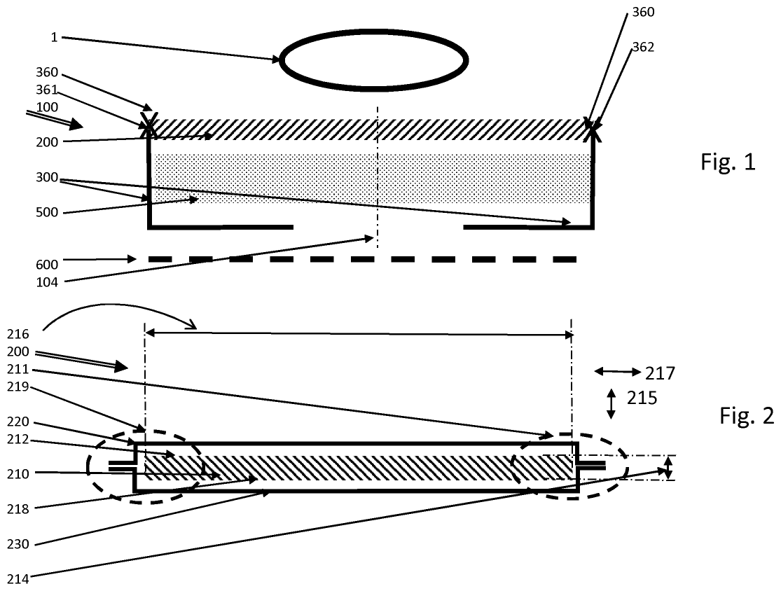

In FIG. 1, a typical and well known set up for the use of an absorbent mat as a bed pad is shown in a cross-directional (i.e. y-z) view. A user (1) is indicated to be positioned on a support (500), such as a mattress, which may be placed on a further support structure (600), such as a slatted frame of a bed. The absorbent mat (100) is shown in the in-use configuration, such that its absorbent composite (200) is positioned between the user (1) and the support (500), whilst the wings (300) are tucked between the support (500) and the further support structure (600). The absorbent mat is exemplarily shown with an even thickness and exhibiting a longitudinally extending centerline, indicated by the dash-dotted line (104).

FIG. 2 depicts schematically the cross-sectional view of an absorbent composite (200), as may be included in the absorbent mat (100), exhibiting a length, width direction (217), and thickness direction (215) which are aligned with the length, width and thickness direction of the absorbent mat, and having a thickness extension (214) substantially smaller than its length and width (216) extension.

The absorbent composite comprises a first surface adapted to be oriented towards a user during use comprising a liquid permeable topsheet (220), and a second, z-directionally opposite surface comprising a liquid impermeable backsheet (230). The liquid permeable topsheet (220) may be positioned on the first surface (212) of the absorbent structure (210) and a liquid impermeable backsheet (230) may be positioned on the second surface (218) of the absorbent structure (210) z-directionally opposite of the topsheet (220). Thusly, the topsheet (220) and the backsheet (230) may envelop the absorbent structure (210) in MD, CD, and thickness direction;

Further, the absorbent composite (200) exhibits a first (211) and a second (219) longitudinally extending lateral side margin region. In the present context, a “lateral side margin region” of the absorbent composite comprises the periphery and proximity of the lateral side margin, that is delimited on one side by the lateral outermost point of the absorbent composite, in an essentially flat state and by a point that is positioned inwardly (i.e. towards the longitudinal centerline) no more than 25% of the total absorbent composite width. A wing is connected in at least a portion of this lateral side margin region of the composite thereto, and may be positioned adjacent thereto or folded around, as will be described in more detail.

For explanation, in FIG. 2, the lateral side margin regions are indicated by ellipses (211 and 219, respectively), comprising the butt seams of the topsheet (220) and the backsheet (230) that extend laterally outwardly from the absorbent structure (210) and extending about 25% of the total width of the absorbent composite towards the longitudinal centerline.

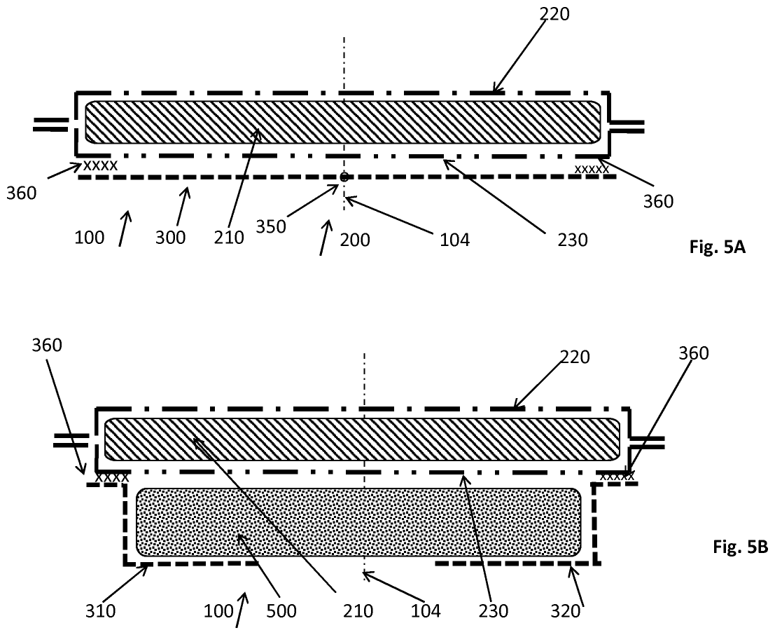

Further executions for the lateral side margin regions are depicted in FIGS. 5 to 12, showing also the wing arrangement in these regions, and discussed in more detail herein below.

As can be further seen exemplarily in FIGS. 3A and 3D for the use configuration, the absorbent mat (100) further comprises a 1^{st} (310) wing, as may be a left wing in a certain view, and a 2^{nd} (320) wing, that might be a right wing in such a view. In FIG. 3A, the wings are wrapped around a support (500), whilst in FIG. 3D the wings are spread out on a flat support (500).

The 1^{st} wing (310) exhibits a longitudinally extending first 1^{st} wing side margin region (312), in an at least partly overlapping positioning with said first longitudinally extending lateral side margin region (211) of said absorbent composite (200), and a longitudinally extending second 1^{st} wing side margin (318) outside of said first 1^{st} wing side margin region (312). The 1^{st} wing is further connected by a connection (361) in the first 1^{st} wing side margin region (312) to the longitudinally extending first lateral side margin region (211) of the absorbent composite (200).

The 2^{nd} wing (320) exhibits a first 2^{nd} wing side margin region (322) in an at least partly overlapping positioning with said second longitudinally extending lateral side margin region (219) of said absorbent composite (200), and a longitudinally extending second 2^{nd} wing side margin (328) outside of said first 2^{nd} wing side margin region (322). The 2^{nd} wing is further connected by a connection (362) in the longitudinally extending first 2^{nd} wing side margin region (322) to the longitudinally extending second lateral side margin region (219) of the absorbent composite (200),

Thereby, the first 1^{st} wing side margin region (312) and the first 2^{nd} wing side margin region (322) are positioned oppositely of the longitudinally extending center line (104) of the absorbent mat.

Whilst it may be preferred from a manufacturing simplicity point of view that the wings exhibit a generally rectangular shape, these may have different shapes such as trapezoidal shape or have curvilinear margins.

The absorbent mat (100) is adapted to be transformed from a manufacturing configuration into a use configuration, as schematically indicated in FIG. 3 A and B, showing a support (500) covered by an absorbent mat. For the manufacturing configuration, the wings 310′ and 320′ are shown in FIG. 3B with dotted lines with their tear-open section 350.

In the manufacturing configuration, at least a portion of the 1^{st} (310) and the 2^{nd} (320) wing extends y-directionally from the first and second side margin regions (312, 322) towards the longitudinally extending centerline (104) of the absorbent mat, respectively. Further, the second 1^{st} wing side margin (318) and the second 2^{nd} wing side margin (328) are connected to each other along in a tear-open section (350) extending essentially parallel to the longitudinal centerline (104). The tear-open section may be any arrangement that maintains the connection during the manufacturing, including confecting and packaging, but which can be readily opened by a user upon use. The user may be the person that will then be positioned on the mat, but may also be a separate person, such as a caretaker in case of bedridden people, or parents in case of baby change mats.

Thus it is preferred that the tear force to tear the connection open does not exceed about 6N, but should be sufficient to withstand process and handling operation, and may be more than about 0.5 N, when assessed according to EDANA test methods WSP 110.4.

In a preferred execution, the tear-open section may be a zone of weakness in the material, that preferably extends over the full length of the wings and intersects the generally cross-directionally extending longitudinal margins of the wings. When applying a pull force at this intersection, the 1^{st} and 2^{nd} wing can be readily separated from each other without damaging the material adjacent to the zone of weakness.

Preferably, such a zone of weakness may be created by a perforation line or partly severed cut line in a web of wing material, from which the two wings are formed. The perforation line may comprise a series of openings in the material arranged in a pattern, typically a linear, and preferably a straight line pattern, such that original material remains essentially unaltered between neighboring openings.

Preferably, the openings are cuts, exhibiting essentially no measurable area, though they may have a 2D extension, such as when holes are punched out of the material.

The perforation seam defines a tear path, such that when a pull force is applied at the intersection of the margin of the wings with the perforation seam, one after the next of the land areas between neighboring openings will give, allowing a clean tear path.

The strength required to open the wings is preferably adjusted so as to allow an average person to open it, and is preferably in the range of 0.5 N to 6 N. A skilled person will be able to readily adjust appropriate strength, such as by varying the perforation seam pattern, especially the distance of neighboring openings. Thus, a perforation line may have a ratio of opening or cut length to land area length along the tear path of from about 50%/50% to about 90%/10%, for example 70%/30%, respectively.

In a second option, the 1^{st} and the 2^{nd} wing are made from different materials or—when being same materials—delivered as separate web materials, which are connected in the tear-open section such that can be re-separated as described for the perforation seam. This may be achieved by overlapping the material and applying adhesive or melt-fusion bonding, such as heat-bonding, that connects the wings with a lower strength than their material strength. It is also contemplated, that a further material is added, as exemplarily and for explanatory, but not limiting purposes, indicated schematically in FIGS. 12A and B, showing an connecting patch 367, which may be firmly or even permanently connected to a 1^{st} wing and releasably or at a lower strength than the 2^{nd} wing. The connecting patch may be executed by comprising adhesives, such as pressure sensitive adhesives, or it may be executed as mechanical fastener, as well known in the art.

In the use configuration, see FIG. 3B, the 1^{st} (310) and the 2^{nd} (320) wings are separated from each other along the tear-open section (350) and at least portions of the second 1^{st} wing side margin (318) and the second 2^{nd} wing side margin (328) are positioned on the backsheet (230) side of the absorbent composite (200), adapted to at least partly wrap around a support (500).

Herein, the term “positioned on the backsheet respectively topsheet side” refers to the configuration that—in Cartesian coordinates, it is z-directionally further away from the absorbent composite than the backsheet or topsheet, respectively. If the material is not arranged in a linear or straight arrangement, at least a portion of the projection of the material onto an x-z, or y-z plane is z-directionally further away from the absorbent composite than the backsheet, as shown in FIG. 3A, or topsheet, respectively.

Referring to FIG. 3B, a perspective view of an absorbent mat is show, wherein the longitudinal extension of the wings is shorten than the one of the absorbent composite. The manufacturing configuration is indicated by dashed line, the arrows (355) indicate the opening action of a user, and the solid lines represent the use configuration.

It should be noted, that it is not important for the present invention, if the wings have the same, shorter or a longer length compared to the absorbent composite.

It is also included in the scope of the present invention that to each side of the absorbent composite two or more wings are connected, as indicated in FIG. 3C with a 1^{st} wing (310′) and a second wing (320′) and a further 1^{st} wing (310″) and a further 2^{nd} wing (320″).

In FIG. 3D a use configuration is indicated, where the absorbent mat is placed on an essentially flat surface as support with the backsheet side towards this surface, and the wings spreading laterally outwardly.

In FIG. 12B the situation is depicted, where the tear-open section is executed with an additional connecting patch (357), that may further be use to re-connect the wings upon wrapping around the support (500).

It should be noted that in the context of positioning the 1^{st} and 2^{nd} wing portions, the term “in an at least partially overlapping positioning” includes a configuration, where a portion of the wing is attached to the topsheet side, but other portions extend around the lateral side margins towards the backsheet side, thereby forming a wrapping of the wing material around the lateral side regions, and thusly a side seal such as against lateral liquid spillage. Having thusly described the key elements of the present invention, which can be used alone or in combination with particular or preferred executions.

In a second aspect, the present invention relates to a manufacturing process for an absorbent mat, comprising an absorbent composite comprising an absorbent structure enveloped between topsheet and backsheet and wings, and exhibiting a length, width, and thickness direction, preferably of an absorbent mat as described in the above.

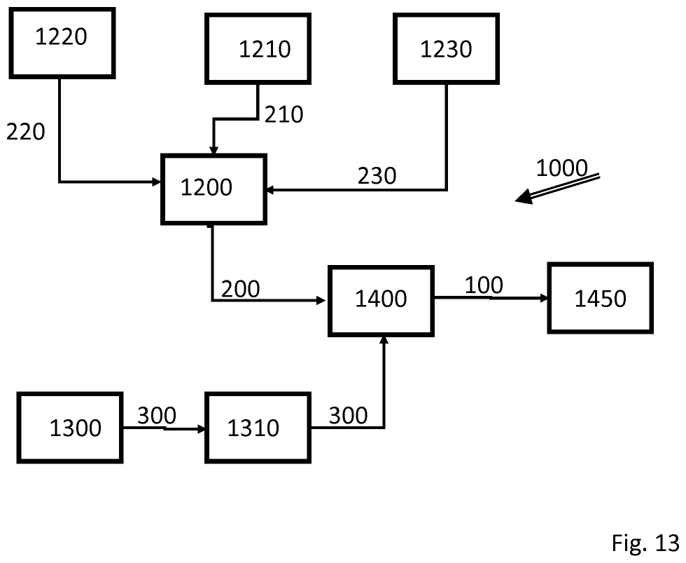

Generally, the process as described in FIG. 13, can be operated on a machine (1000) exhibiting a machine (MD) (1010) and a cross-machine (CD) direction, and a z-direction perpendicular thereto, whereby MD is aligned with the length direction of the absorbent mat.

The process further comprises providing of materials from respective supply means, namely of an absorbent structure (210), preferably a preformed absorbent web material, a topsheet (220) and a backsheet (230) web material, and a web of wing material (300) for forming the 1^{st} and the 2^{nd} wings preferably as a unitary material at a width of at least the sum of the width of the wings.

The process is operated on an equipment (1000) that comprises supply for the web materials, namely for the absorbent structure (1210), the topsheet (1220), the backsheet (1230), and the wing material (1300). Such supply means are known in the art such as unwind stands for rolls or spools, or so called de-festooner for feeding web materials out of a box. Also guide means for the materials are well known (not shown).

In a material combining unit (1200) the absorbent composite can be formed, and the equipment further comprises a tool for applying the tear-open section (1310), such as a separating or perforating means for applying zones of weakness, such as partly separating or perforating the web of wing material in a machine direction, such as conventional rotary knives, or water jets, all also well known in the art. Preferably, the separation is executed such that no hard edges are created, and most preferably the equipment is a rotary knife. The combining of the wing material and the absorbent composite to form the absorbent mat can be executed in the mat combining unit (1400), which may be followed by confecting means (1450), which my comprise as final cutting units for separating—partly or completely—machine directionally neighboring mats, further folding or winding units and packaging units.

The process is generally executed as a continuous process with a continuous process base web. This process base web may be the absorbent composite, a backsheet material when discrete pads are placed on a longer backsheet, but also the wing web material, if the length of the wings exceed the length of the absorbent composite. At the end of the process, the continuous process base web is typically cross-directionally separated (“final cut”) to form individual absorbent mat articles. However, it is also contemplated, that the absorbent mats remain connected, and may be rolled on a roll, or be festooned into a box, though preferably comprising a perforation or separating line or region between two machine-directionally neighboring mats.

Thus, the process for the manufacture of an absorbent mat, comprises the following process steps:
Providing the materials from the supply units (1210, 1220, 1230, 1300), respectively.
Forming an absorbent composite as may be performed by enveloping separated pieces of a continuous absorbent web material as absorbent structure between a topsheet and a backsheet, thereby forming an essentially continuous series of absorbent composites (200) exhibiting a first (211) and a second (219) longitudinally extending lateral side margin region. Alternatively, the absorbent composite may be formed from individually formed absorbent structures, such as made on well-known core former, enveloped between a topsheet and a backsheet. The topsheet or the backsheet or both may already be integrally form with the absorbent structure, though for this options preferably additional measures are taken to provide clean side edges without spillage of any material of the absorbent structure, as can be achieved by embossing or application of adhesives to the margins or by overfolding wings around the side margins as side seals not only preventing liquid spillage, but also of materials of the absorbent composite.
Creating a tear-open section (350) in the wing material (100) generally parallel to the machine direction, as may be by the application of a perforation seam, that may be a straight line parallel to the longitudinally extending centerline, or it may be curved, such as sinusoidal, line.
Selecting least one of
   a web of wing material and
   a web comprising an absorbent structure
as a continuous base web.
Optionally separating one of
   the web of wing material and
   the web comprising an absorbent structure
into machine-directionally individualized pieces.
Feeding the selected continuous base web or webs and the individualized pieces, if present, to a mat combination unit.
Positioning the selected continuous base web or webs and the individual pieces, if present, in the mat combination unit, such that the first 1^{st} and 2^{nd} wing side margin regions (312 and 322, respectively) are positioned in an at least partly overlapping positioning with the first and second longitudinally extending lateral side margin region (211 and 219, respectively) of the absorbent composite (200), and such that the longitudinally extending second 1^{st} and 2^{nd} wing side margin (318 and 328, respectively) are outside of said first 1^{st} and 2^{nd} wing side margin regions (312, 322, respectively).
Connecting the longitudinally extending side margins of the wing material and the longitudinally extending side margin regions of the absorbent composite, wherein the connecting (361, 361, respectively) is preferably executed by the application of adhesives or one or more energy sources selected from the group consisting of heat, pressure, friction, and ultrasonics.
separating said continuous base web into said absorbent mats, optionally executing further processing steps, preferably comprising folding, and optionally comprising packaging,
wherein the tear-open section in the wing material remains essentially intact.

Thus, in a first option of this process the continuous base process web material is a continuous web of wing material and a piece of the absorbent composite is added thereto. In a second option of this process, the continuous base process web material is a web of absorbent material and a piece of the win material is added thereto. In yet a third option, both the absorbent composite and the wing exhibit the same length in the product, and either of the two or both may be supplied as continuous base web material.

Whilst the process allows for positioning the wing such that it is on the either the topsheet or the backsheet side of the absorbent composite, it is a preferred option to position it on the topsheet side.

In a third aspect, the present invention relates to the use of an absorbent mat as described in the above and a method of applying it. To this end, the absorbent mat comprising an absorbent composite and wings that are connected to each other by a tear-open section, such as a perforation, that is opened by a user, such by tearing the perforation open. Then the wings are folded outwardly, as indicated by arrows (355) in FIGS. 3B and C. Optionally and often preferably, the absorbent mat is placed onto the support, such as a mattress, prior to the opening, which would enable easier and cleaner application, in particular for the option of the wings covering the topsheet of the absorbent composite.

Depending on the support and its potential arrangement relative to a further support structure, the wings may then be attached to the support or further support structure, be it by being tucked between these, and/or by using the optional further fastener, such as mechanical fastener, for connecting the wing to each other, to the support, or the further support structure.

Further to the general description of the present invention in the above, the following will now describe particular executions for the present invention by referring to FIGS. 5 to 10, 12, and 14, showing a manufacturing configuration (Fig. n.A) and a representative use configuration (Fig. n.B), respectively. It should be noted, that with a support different that the rectangular one as indicated in the figures, this configuration may look different.

In all figures, the tear-open section for connecting the 1^{st} and 2^{nd} wings is indicated by numeral 350, whilst the connecting of the wings and the absorbent composite is indicated by numeral 360 for a first and 365 in case of a second connecting for each of the wings.

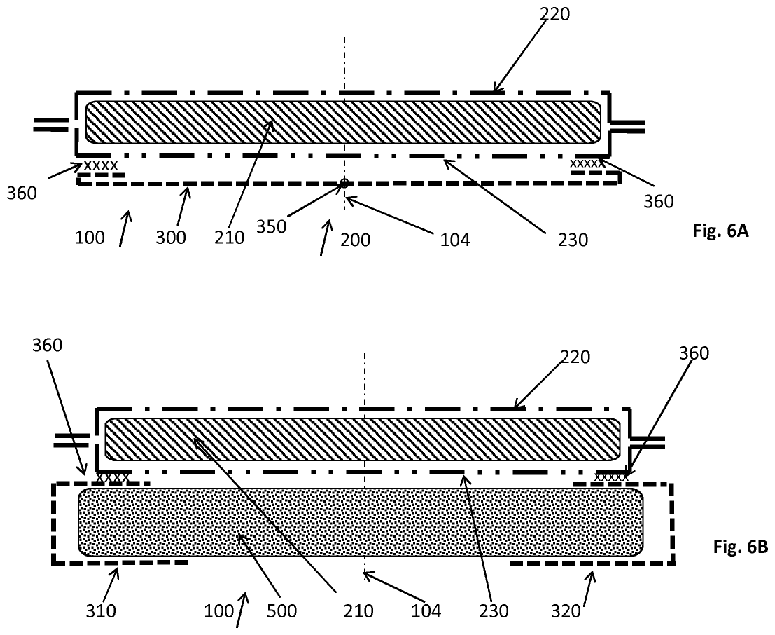

In FIGS. 5A and B, a first embodiment is described, with a manufacturing configuration where the wings are being positioned on the backsheet side of the absorbent composite. It should be noted, that in this particular design, the “butt seam” of the wings with the absorbent composite may create peel force stress at the connection 360 such that in a preferred execution this connection is a melt fusion connection rather than an adhesive connection, such as a particularly preferred bonding as described in WO2012/042055A1. FIG. 6 A and B depict an arrangement similar to the one as shown in FIG. 5 with the wing being inwardly folded on the backsheet side in the lateral side margin region in the manufacturing configuration.

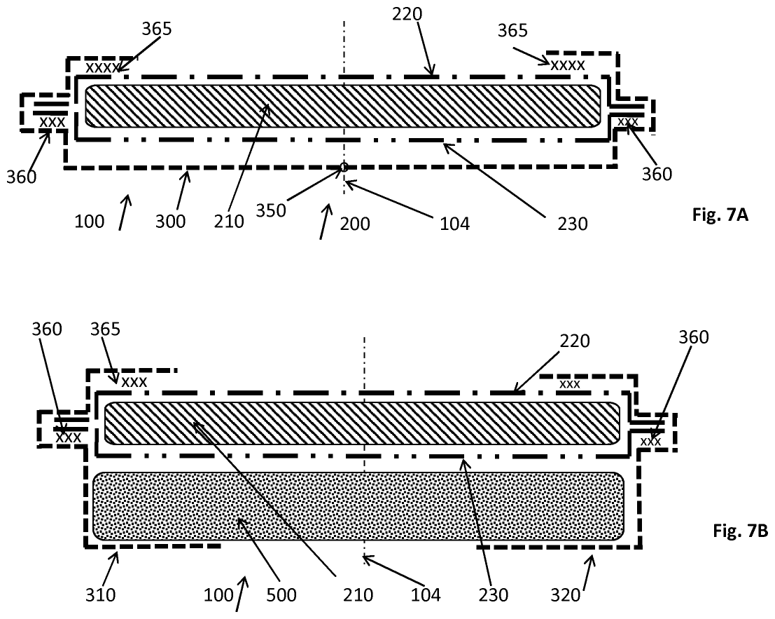

FIGS. 7A and B depict a further arrangement, with the wings being positioned on the backsheet side of the absorbent composite. However, the wings are folded around the lateral margin of the absorbent composite and extend in the lateral side margin region into the topsheet side, where these may be connected to the topsheet by an additional connection (365). Such a side seal arrangement is particularly useful, when the wings are executed as liquid impermeable material, as lateral spreading of any liquid at the side is reduced or prevented. Preferably, at least the surface of the wings that is positioned towards a user during use is made of a skin friendly material, such as a nonwoven material, such as a film/nonwoven laminate known as “textile backsheet” e.g. for diapers, or a hydrophobic nonwoven.

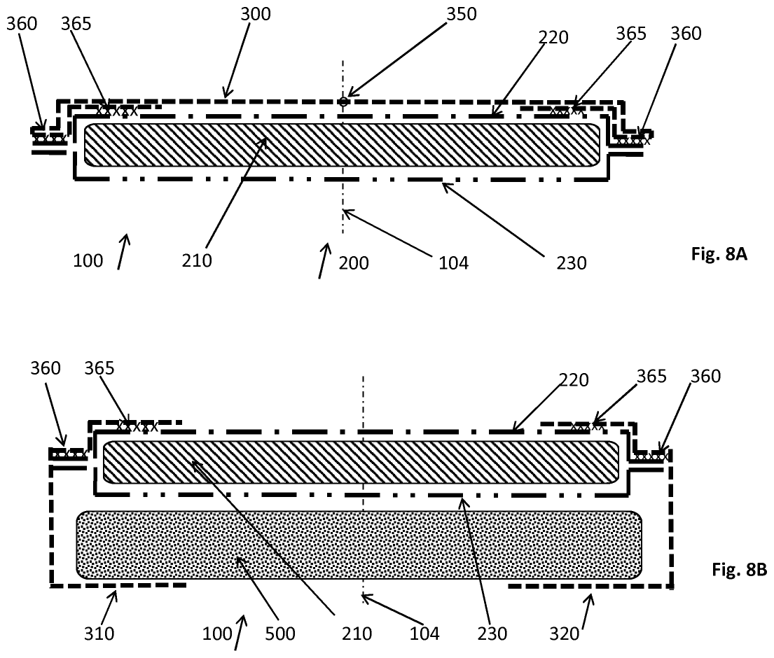

FIGS. 8A and B depict a further and often preferred arrangement. Compared to the one of FIG. 7, the wings are folded over to cover the full topsheet side of the absorbent composite in the manufacturing configuration, whilst maintaining in the use configuration the protection of the lateral side margin regions. As shown, a further connection of the wing to the absorbent composite (365) is made such that the wing material is connected to the topsheet material.

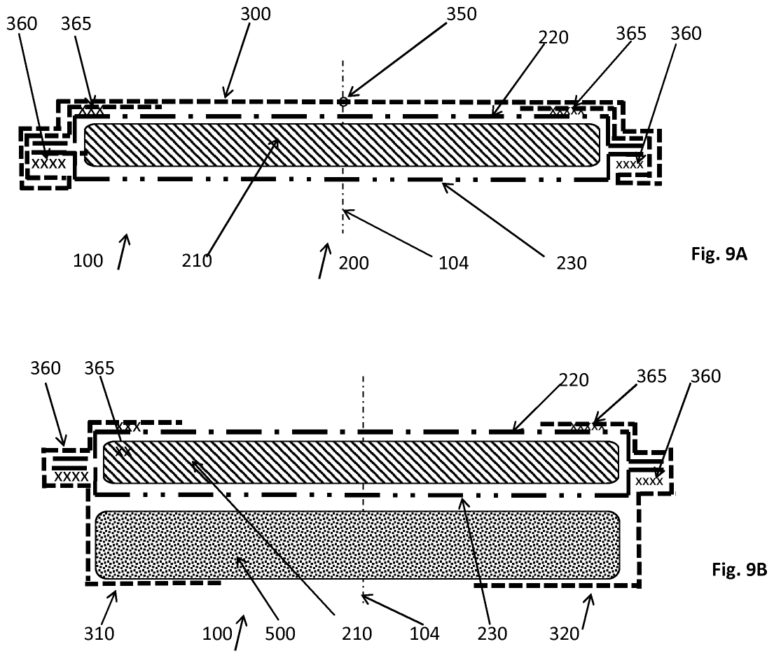

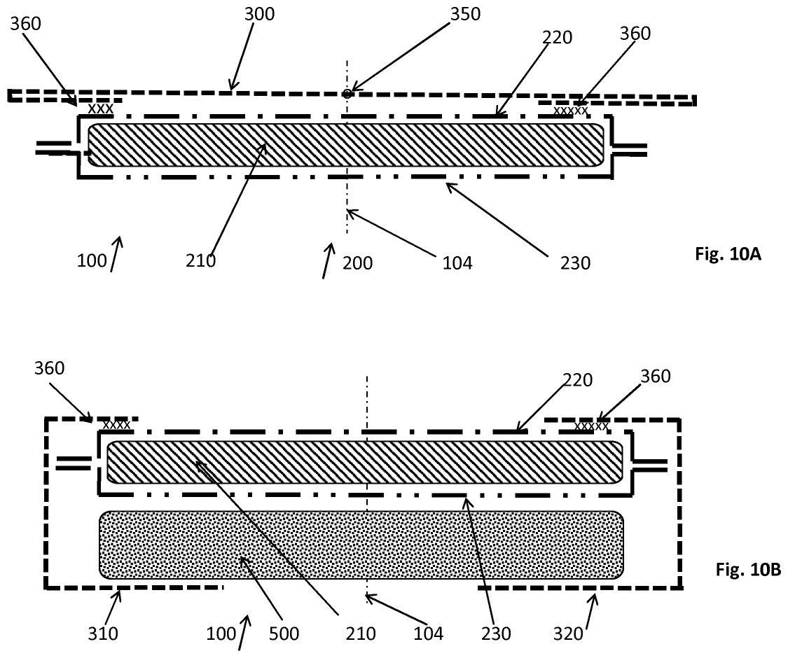

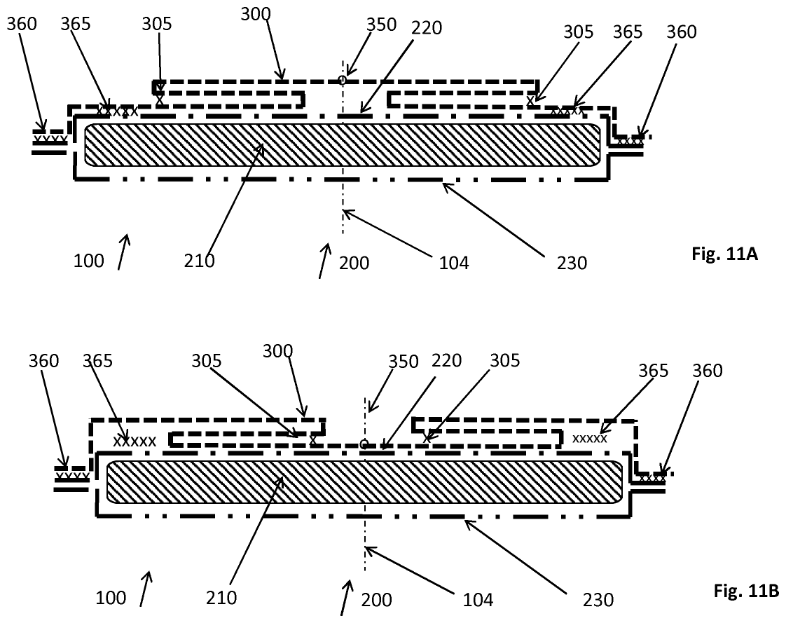

FIGS. 9A and B depict a very similar design compared to the one of FIG. 8 A and B, except that the wing material is folded around the side margins and is connected to the backsheet material.

FIG. 10 represents a further variant, wherein the topsheet of the absorbent composite is covered by the wing in the manufacturing configuration. Further the wings provide a side seal against lateral leakage by being—when executed as a liquid impermeable material—folded around the lateral side margins of the absorbent composite in the use configuration. As indicated in FIG. 10, it might be desirable to have a width of the wings other than about half the width of the absorbent composite.

Yet a further execution for designing a side seal wrapping with the wings is depicted in FIG. 14, showing the manufacturing configuration, wherein the wing material (300) is folded around the lateral edges of the topsheet (220) absorbent structure so as to envelop the topsheet in the side seal region (211, 219). The wing protects against side leakage from the absorbent core and this sealing is further improved by the adhesive layer (365), that may also connect the topsheet to the backsheet, such that the liquid impermeability requirement for the wing material may be reduced.

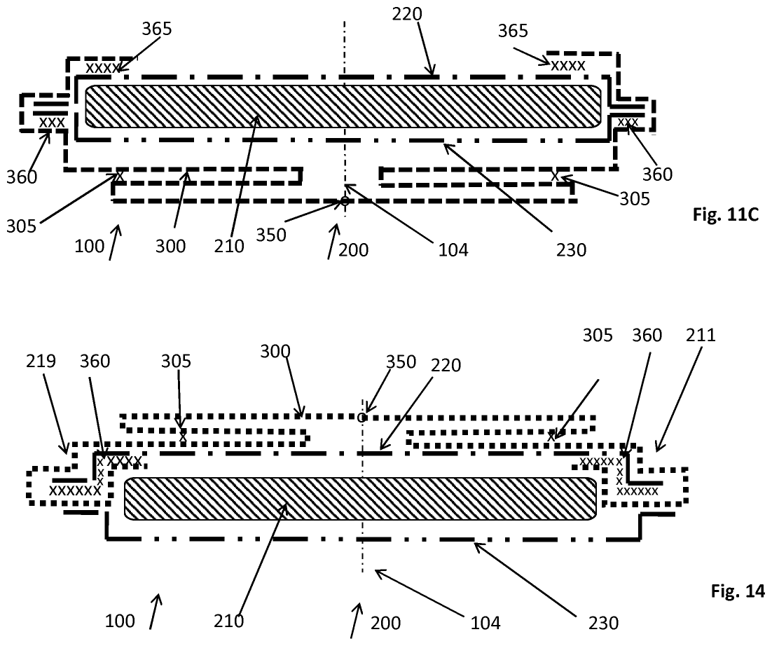

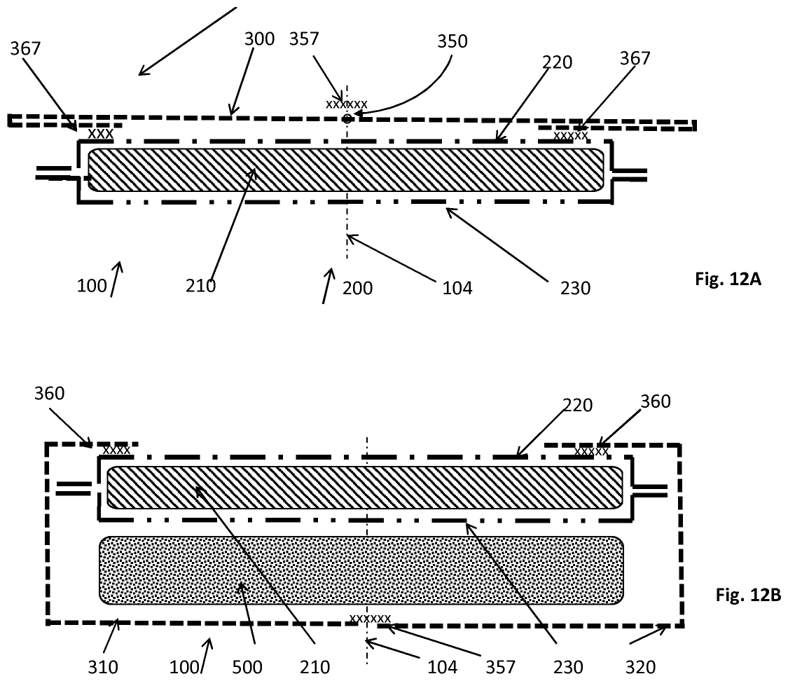

Henceforth, an execution as indicated in FIG. 11 A and B as a particular modification for the manufacturing configuration as shown in FIG. 8A offers further flexibility in allowing to fold the wing material in a zig-zag or even leporello fold, either outwardly, i.e. the further folding being further away from the topsheet, see FIG. 11A, or inwardly, i.e. the further folding is positioned towards the topsheet, see FIG. 11B. FIG. 11C shows that this principle can be evenly applied to wings positioned on the backsheet side of the absorbent composite, as exemplarily shown in a modification of the design of FIG. 7.

In any of the executions, and in particular in the ones as exemplified in FIG. 11 A to C, a fold fixation (305) may optionally be applied, such as by a glue dot or bead, primarily to ease processing without hindering use and application. To this end, this connection exhibits preferably only a small tear force, such as of less than about 0.2 N, when measured according to EDANA test method WSP 110.4.

In FIG. 12, it is indicated, that a further releasable connecting means, such as tapes with pressure sensitive adhesive or mechanical fastener, may be applied to the wing in the tear-open section. As shown exemplarily, the further connecting means (357) may be placed over a complete or partial separation line (350) in the manufacturing configuration. Upon use, as indicated in FIG. 13B, the further connecting means may be used to re-connect the wings to themselves underneath the support (500) or directly to the support (not shown).

1. An absorbent mat (100) for being positioned on a support (500) for a person (1) as a user,
said absorbent mat (100) exhibiting in Cartesian coordinates
a longitudinal or length (x-) direction,
a width (y-) direction,
a thickness (z-) direction perpendicular thereto
a longitudinally extending center line (104);
said absorbent mat (100) comprising an absorbent composite (200) comprising
(i) an absorbent structure (210)
exhibiting a length, width (217), and thickness (215) direction aligned with the length, width and thickness direction of said absorbent mat,
and having a thickness extension (214) substantially smaller than its length and width (216) extension;
and a first surface (212) adapted to be oriented towards a user during use,
and a second, opposite surface (218);
(ii) a liquid permeable topsheet (220) positioned on said first surface (212) of said absorbent structure (210);
(iii) a liquid impermeable backsheet (230) positioned on said second surface (218) of said absorbent structure (210) z-directionally opposite of said topsheet (220);
and said absorbent composite (200) exhibiting a first (211) and a second (219) longitudinally extending lateral side margin region positioned oppositely of said longitudinally extending center line (104) of said absorbent mat,
said absorbent mat (100) further comprising a 1^{st} (310) and a 2^{nd} (320) wing
said 1^{st} wing (310)
exhibiting
a first 1^{st} wing side margin region (312)
in an at least partly overlapping positioning with said first longitudinally extending lateral side margin region (211) of said absorbent composite (200),
and a longitudinally extending second 1^{st} wing side margin (318) outside of said first 1^{st} wing side margin region (312),
and said 1^{st} wing (310) being connected by a connection (361)
in said first 1¹ wing side margin region (312)
to said longitudinally extending first lateral side margin region (211) of said absorbent composite (200),
said 2^{nd} wing (320)
exhibiting
a first 2^{nd} wing side margin region (322)
in an at least partly overlapping positioning with said second longitudinally extending lateral side margin region (219) of said absorbent composite (200),
and a longitudinally extending second 2^{nd} wing side margin (328) outside of said first 2^{nd} wing side margin region (322),
and being connected by a connection (362)
in said first 2^{nd} wing side margin region (322)
to said longitudinally extending second lateral side margin region (219) of said absorbent composite (200),
wherein said absorbent mat (100) is adapted to be transformed from a manufacturing configuration
wherein at least a portion of said 1^{st} (310) and said 2^{nd} (320) wing extends y-directionally from said first and second side margin regions (312, 322) towards said longitudinally extending centerline (104) of said absorbent mat, respectively,
and wherein said second 1^{st} wing side margin (318) and said second 2^{nd} wing side margin (328) are connected to each other along a tear-open section (350) extending essentially parallel to said longitudinal centerline (104),
into a use configuration,
wherein said 1^{st} (310) and said 2^{nd} (320) wings
are separated from each other along said tear-open section (350)
and at least portions of
said second 1^{st} wing side margin (318) and
said second 2^{nd} wing side margin (328)
are positioned
(a) on the backsheet (230) side of said absorbent composite (200),
or
(b) laterally outwardly of said first and second longitudinally extending lateral side margin regions (212, 218), respectively.

2. An absorbent mat according to claim 1, wherein said tear-open section is a zone of weakness, extending generally in the length direction, and more preferably a straight or curved perforation line.

3. An absorbent mat according to claim 1, wherein said connecting of said wings (300) to said absorbent composite is selected from the group consisting of adhesive, heat-, or ultrasonic bonding.

4. An absorbent mat according to claim 3, wherein said connecting is executed as line(s), dots, or regions.

5. An absorbent mat according to claim 1, wherein said absorbent composite (200) is pre-formed.

6. An absorbent mat according to claim 1, wherein in said manufacturing configuration said tear-open section (350) of said wings is positioned on the topsheet side of the absorbent composite.

7. An absorbent mat according to claim 1, wherein in said manufacturing configuration said tear-open section (350) of said wings is positioned on the backsheet side of said absorbent composite.

8. An absorbent mat according to claim 1, wherein the wings are zig-zag or leporello folded, optionally comprising a fold fixation (305) for such folds, preferably an adhesive dot or line.

9. An absorbent mat according to claim 1, wherein said wings (300) are connected to said absorbent composite (200) in said longitudinally extending first 1^{st} wing side margin region (312) and said first 2^{nd} wing side margin region (318) to the topsheet (220) of said absorbent composite (200).

10. An absorbent mat according to claim 9, such that said wings are adapted to form a side seal wrapping of said absorbent composite (200) in the use configuration.

11. An absorbent mat according to claim 1, wherein said wings (300) are essentially made from liquid impermeable but air permeable materials, preferably hydrophobic nonwoven.

12. An absorbent mat according to claim 1, wherein said support is a mattress.

13. A method of applying an absorbent mat according to claim 1 to a support, said method comprising the steps of separating said 1^{st} and said 2^{nd} wing from each other along said tear-open section and optionally at least partly wrapping said support by said wings.

14. An absorbent mat according to claim 1, whereby said topsheet (220) and said backsheet (230) are enveloping said absorbent structure (210) in MD, CD, and thickness direction.

15. An absorbent mat according to claim 1, wherein said tear-open section is a zone of weakness, extending generally in the length direction, is a straight or curved perforation line.

16. An absorbent mat according to claim 1, wherein the wings are zig-zag or leporello folded and comprise a fold fixation (305) for such folds.

17. An absorbent mat according to claim 1, wherein the wings are zig-zag or leporello folded and comprise an adhesive dot or line as fold fixation (305) for such folds.

18. An absorbent mat according to claim 1, wherein said wings (300) are essentially made from hydrophobic nonwoven.

19. A method of applying an absorbent mat to a support according to claim 13, said method further comprising the step at least partly wrapping said support by said wings.
